(19) United States
(12) Patent Application Publication
(10) Pub. No.: US 2011/0144768 A1
(43) Pub. Date: Jun. 16, 2011

(54) Device and method for securing an attachment device for a prosthesis and/or a prosthesis adapter to a prosthesis shaft and protective lining

(75) Inventors: Dirk KREMSER, BAYREUTH (DE); Christof KURTH, HEINERSREUTH (DE)

(73) Assignee: MEDI GMBH & CO. KG, BAYREUTH (DE)

(21) Appl. No.: 12/963,788

(22) Filed: Dec. 9, 2010

(30) Foreign Application Priority Data
Dec. 11, 2009 (DE) 10 2009 057 897.8

(51) Int. Cl. A61F 2/78; A61F 2/80

(52) U.S. Cl. 623/33; 623/36

(57) ABSTRACT

Device (1) for securing an attachment device (13) for a prosthesis and/or a prosthesis adapter (37) to a prosthesis shaft (11), comprising:
a first holder (10) for the prosthesis shaft (11),
a second holder (12) for the attachment device (13), said second holder (12) being arranged in particular below the first holder (10),
means for relative sliding and/or pivoting of the prosthesis shaft (11) fixed in the first holder (10) and of the attachment device (13) fixed in the second holder (12), and
means for firmly connecting the fixed prosthesis shaft (11) and the fixed attachment device (13) in a desired relative position and/or orientation.

The invention relates to a device and a method for securing an attachment device for a prosthesis and/or a prosthesis adapter to a prosthesis shaft, and an associated protective lining.

In prostheses today, it is known to first connect an attachment device to the prosthesis shaft. An attachment device for a modular prosthesis adapter is often used, such that it is possible to attach different prostheses.

Procedures for connecting a prosthesis shaft to such an attachment device are known in the prior art, especially for leg prostheses.

There are basically two different ways of connecting the prosthesis shaft to the distal adapter parts.

One possibility is to connect a prosthesis adapter directly to the prosthesis shaft. In the case of interim prosthesis shafts made of plastic, it is known to adhesively affix and/or screw on adapters. In the case of permanent shafts, in particular those made from laminated composite materials, the adapter can be encapsulated, for example, and cast. This type of connection has the disadvantage that it is suitable only for relatively straight amputation stumps with minimal flexion contracture. In cases of greater flexion contractures, it is no longer possible to ensure a correct static prosthesis set-up by the twice tilting of modular connections. The point of the prosthesis attachment must then be mounted on the shaft in a position offset horizontally toward the stump end.

Therefore, a connection by means of a prosthesis adapter is suitable only for amputees who require a relatively flexion-free (straight) prosthesis set-up. The use of additional adapters that change the position of the prosthesis shaft in the horizontal direction results in a heavier weight and a higher cost and of course, more work.

A further disadvantage of this described first known method is that the height at which the prosthesis adapter is arranged cannot be varied. The prosthesis adapter is in principle fixed directly under the prosthesis shaft. Especially when constructing above-knee prostheses, it is sometimes necessary for the adapter attachment point to be arranged on the shaft in a position offset in the distal direction, in order to be able to directly secure the prosthetic knee joint, without the use of additional adapters, in such a way that the rotation point of the prosthetic knee joint is located at the height of the rotation point of the knee on the contralateral side.

For this reason, in a second conventional way of setting up prosthesis shafts, the shaft is lengthened by polyurethane foam (PU foam) being cast on. The hardened foam is then shortened in such a way that the adapter can be affixed to the foam in a position whose tilting and sliding in the x, y and z axes is suitable for the prosthetic set-up.

This connection is generally produced in the following way: the prosthesis shaft is turned over, and a conical or cylindrical sleeve is fitted onto the distal end. Thereafter, a PU foam to be mixed together from two components is cast in liquid form into the enclosed area. After the PU foam has expanded and hardened, it is ground down to the desired height and shaft flexion position. An attachment device is then secured in the desired position to the foam. This connection is normally obtained by adhesive bonding. If it is necessary to make a relatively large change to the horizontal position of the attachment device, the PU foam can also be easily cut through and joined together again in another position.

However, the durability of this connection or this securing method is not very great, and it is therefore necessary to strengthen this connection in order to permit longer use of the prosthesis by the amputee. This is done by subsequently applying an outer reinforcing layer, for example laminate layers or plastic binding.

This connection or procedure has the following disadvantages. First, the correct position of the prosthesis shaft and the suitable height of the attachment device are very difficult to establish in this procedure, since the desired relative position and orientation of prosthesis shaft and attachment device is reached only slowly by grinding.

When making the necessary adjustment, the orthopedic technician has to carry out grinding in stages and has to check from outside whether the ideal relative position and orientation are reached. This is not only very time-consuming and difficult, it is also not easy to document.

A crucial factor for the durability of the connection between the prosthesis shaft and the attachment device is, among other things, also the correct mixing ratio of the PU foam, which is dependent on the orthopedic technician. The manually produced connections are often of relatively poor quality. In addition, the adhesive connection between the attachment device and the PU foam forms an undesired predefined break point.

Therefore, the object of the invention is to specify a method and a device for connecting a prosthesis shaft to an attachment device, by which method and device it is easily possible to make very precise adjustments to various geometries, in particular the relative position and orientation of prosthesis shaft and receiving device.

According to the invention, a device provided to achieve this object of securing an attachment device for a prosthesis and/or a prosthesis adapter to a prosthesis shaft is characterized by:
a first holder for the prosthesis shaft,
a second holder for the attachment device, said second holder being arranged in particular below the first holder,
means for relative sliding and/or pivoting of the prosthesis shaft fixed in the first holder and of the attachment device fixed in the second holder, and
means for firmly connecting the fixed prosthesis shaft and the fixed attachment device in a desired relative position and/or orientation.

According to the invention, it is therefore proposed to make available a dedicated device in which the prosthesis shaft and the attachment device can first be fixed in respective first and second holders. By means of means for relative sliding and/or pivoting of the suitably positioned parts to be connected, a desired final relative position and/or orientation that is ideal for the patient can then be comfortably set to the precise height. After this has been done, the means for the firm connection provided according to the invention can be used to create a firm connection between the prosthesis shaft still fixed in the first holder and the attachment device fixed in the second holder. This means that while the connection is being made, which connection thus finally secures the attachment device to the prosthesis shaft, the desired relative position and/or orientation is maintained in each case, such that complex readjustment or the like is also entirely unnecessary. Depending on the final relative position and/or orientation, the connection can entail indirect securing or in many cases also direct securing, preference being given in the present case to the connection being produced using a filler, as will be discussed in more detail below.

With the device according to the invention, it is therefore possible for the first time to make available an apparatus with which, by producing an exact final relative position and/or orientation, a connection between a prosthesis shaft and an attachment device for a prosthesis and/or a prosthesis adapter can be created in a simple, quick and reproducible manner, such that the needs of an amputee in respect of the prosthesis statics can be met, and a basis is provided for a reliable, stable and correctly oriented prosthesis, i.e. a prosthesis that is comfortable to use.

For example, in the case of an above-knee prosthesis, it is particularly advantageous if the first holder is arranged above the second holder. Provision can then be made, for example, for the amputee to lower his prosthesis shaft into the first holder, where it is fixed. In doing so, it is possible, for example, to choose a predefined position and/or orientation. For example, the prosthesis shaft can first be oriented vertically and can be located in an exactly central position above the horizontally oriented receiving device fixed in the second holder. Then, if appropriate, adjustments can be made if it is necessary to depart from this predefined position and/or orientation. Although the example of a leg prosthesis is often referred to below, it should be emphasized that the device according to the invention is in principle also suitable for other prostheses.

As has already been indicated, it is particularly advantageous if the first holder is designed for the substantially vertical fixing of the prosthesis shaft, since, particularly in the case of a leg prosthesis, the longitudinal direction of the prosthesis then corresponds to the vertical, which is also the preferred orientation of the prosthesis hereinbelow, particularly as regards statics.

In a further embodiment of the present invention, provision can be made that the means for the firm connection comprise a flexible connecting sleeve that can be secured in particular on the attachment device, in particular by means of a web corresponding to a groove of the attachment device, and that seals off the free space between the prosthesis shaft fixed in the first holder and the attachment device fixed in the second holder. This connecting sleeve particularly advantageously serves several purposes, in particular if it is already used before the relative sliding and/or pivoting. On the one hand, it forms a mold for a filler, in particular a PU foam, which is to be used later to form the connection and which, by virtue of its effect of sealing the free space, remains in the free space provided for the connection. The connecting sleeve is flexible, such that a relative sliding and/or pivoting of the fixed parts also remains possible. On the other hand, the connecting sleeve forms a visual indicator of the subsequent appearance of the connection, such that the user seeking the correct relative position and/or orientation, in particular an orthopedic technician, is also provided with a visual indicator allowing him to perform a more precise and more exact adjustment of the future statics of the prosthesis. It is of course particularly advantageous here if the connecting sleeve has a configuration corresponding to the desired final shape of the connection, in particular a frustoconical shape and/or a cylindrical shape. Then, on the one hand, after the introduction and hardening of the filler, which forms the connection, no subsequent working needs to be carried out any more, and, on the other hand, the visual clue provided to the user by the connecting sleeve is improved. It is conceivable advantageously to use both substantially cylindrical shapes, i.e. tubular shapes, and also the shape of a truncated cone, i.e. a slightly conically tapering shape.

As has already been mentioned, it is possible to secure the connecting sleeve to the attachment device, in which respect it will be noted that, although securing is of course also conceivable elsewhere, securing to the attachment device is nonetheless particularly recommended if the second holder is arranged below the first holder, since the main sealing effect is then needed there. For this purpose, the attachment device can, for example, comprise a groove, in particular all the way round the outer circumference of the attachment device, in which case the attachment device expediently has a substantially round shape. A web corresponding to this groove is formed integrally on the edge of the connecting sleeve and, when the connecting sleeve is pushed on and/or rolled on, this web engages in particular with an exact fit in the groove. The securing can be improved, for example, by adhesive bonding. The height of the connecting sleeve is generally chosen such that different distances between the attachment device and the prosthesis adapter can be adjusted, as may be needed in different patients, and yet the free space still remains completely covered.

As has already been mentioned, the means for the firm connection can also be designed such that the free space sealed by the connecting sleeve can be filled with a filler, in particular a PU foam, which then hardens. For this purpose, the means for the firm connection can comprise an injection device and a hose and/or a tube for feeding the filler into the free space. Although provision can of course be made in principle for the hose or tube for feeding the filler and/or a corresponding air removal device to be simply pushed in from above in order to establish a connection to the free space, for example pushed in between prosthesis shaft and connecting sleeve, it is nonetheless particularly advantageously possible for the connecting sleeve to be already designed for this function, the connecting sleeve then being regarded in particular as part of the securing device according to the invention.

Thus, the connecting sleeve can have an inlet opening for introducing a filler, in particular a PU foam, that connects the prosthesis shaft to the attachment device. On the inlet opening, an attachment can additionally be provided for a hose delivering the filler and/or for a tube delivering the filler which, as has been described above, can be part of the means for the firm connection. The inlet opening can be arranged in the lower area, particularly in the state of use of the connecting sleeve. The inlet opening can particularly advantageously comprise a closure device, in particular based on an elastic force, such that the inlet opening can be opened from the outside by a nozzle, for example, and closes again automatically when the nozzle is removed. If a two-component filler, for example a two-component PU foam, is provided as filler, a mixer cartridge can expediently be provided in the hose and/or tube for the purpose of mixing the two-component filler. However, it is of course also conceivable for the filler to be mixed in the injection device. Finally, however, it is also conceivable to mix the two-component filler in a separate container and then introduce it into the injection device. Mixing in the injection device can be achieved by manual shaking, although it is equally conceivable to provide an automatic mixer as part of the injection device. In the context of the present invention, a one-component filler can of course also be used in principle.

The connecting sleeve can also particularly advantageously have at least one air outlet opening, in particular arranged higher than the inlet opening, for letting air out during introduction of the filler. For this purpose, an air outlet is also already integrated in the connecting sleeve, such that an additional hose to be inserted at the upper end for example, as has been described, is no longer necessary. Several air outlet openings can preferably be provided which, in particular, are distributed uniformly and are of a size permitting escape of air and preventing escape of filler. For example, small holes, which can be arranged in circles at different heights of the connecting sleeve, allow air to flow out but, by virtue of their being small, do not allow the filler (connecting compound) to flow out. When the filler reaches the holes or air outlet openings, the latter are closed by the filler. In an alternative embodiment, however, provision can also be made that the air outlet openings are connected to at least one channel which runs inside the connecting sleeve and which is connected to the environment in particular via at least one further opening. For example, these channels can also extend substantially vertically in the installed state. In this case too, the air outlet openings are advantageously of a size that permits the escape of air and prevents the escape of filler, and, consequently, they can then also be closed by the filler.

The connecting sleeve is expediently made of a material that does not undergo any appreciable adhesive bonding to the filler. Then, after the filler has been introduced and has hardened, the connecting sleeve can be removed again and therefore continues to be available for further uses as part of the device according to the invention. This is particularly desirable if inlet and outlet openings are integrated in the construction of the connecting sleeve. However, it will already be noted here that, in an alternative embodiment, the connecting sleeve is designed as a lining that remains part of the connection, in particular as a protective and/or reinforcing lining, and therefore, after the filler has been introduced and has hardened, the connecting sleeve remains as part of the connection between prosthesis shaft and attachment device. However, it may be more expedient for the connecting sleeve to be removable since, if the relative position and orientation are still unfavorable, the connection can, for example, be sawn off and once again established.

In another advantageous embodiment of the present invention, provision can be made that the means for the relative pivoting and/or sliding comprise a height-adjusting mechanism for the first holder and/or at least one pivoting device for the first holder. In this way, the first holder, and therefore the prosthesis shaft, can be moved away from or moved closer toward the plane of the attachment device fixed in the second holder, for example in order to be able to adjust the correct position of a knee joint to the height of the healthy knee joint. In addition, a pivoting device for the first holder can be provided which, in particular, permits pivoting about any desired horizontal axes if the prosthesis shaft is fixed in a substantially vertical orientation. In this way, flexion contractures or the like can be compensated, for example. Such an embodiment can be ideally supplemented if, in particular, the means for the relative pivoting and/or sliding additionally comprise an adjustment mechanism for the horizontal sliding of the second holder relative to the first holder, such that, for example after the vertically fixed prosthesis shaft in the first holder has been pivote about a horizontal axis, it is also advantageously possible to adapt the position of the attachment device perpendicularly with respect to the orientation of the prosthesis shaft.

The height-adjusting mechanism can, for example, have a stand, constructed from hollow profile tubes, with a vertically adjustable rod which is guided therein and which supports the first holder. The first holder can be fixed at a defined height by a screw that engages on the rod. In addition to having a hollow tube in which the vertically adjustable rod is guided, the stand can, for example, have a base-plate on which the second holder is then arranged, if appropriate being able to be moved horizontally via the adjustment mechanism. The stand and the base-plate thus connect the first holder and the second holder, which holders can therefore be changed, in terms of their position and/or orientation relative to each other, only in a defined manner via the means for the relative pivoting and/or sliding. In one embodiment, provision can also be made that the first holder is guided rotatably on the rod by a releasable clamp device as part of the pivoting device. In this way, for example, a pivoting movement can be effected about a vertical axis, if the prosthesis shaft is fixed vertically. However, such a pivoting movement will not be necessary in every case, since such a movement can also be reproduced by the adjustment mechanism for the horizontal sliding of the second holder relative to the first holder.

Preferably, however, the pivoting connection can comprise an articulated bearing of the first holder, in particular an articulated connection, arranged between the first holder and the clamping device, for the pivoting about at least one vertical axis. An articulated connection of this kind can be designed, for example, as a ball joint.

Other means for forming the adjustment mechanism, the pivoting device and the height-adjusting mechanism are widely known in the prior art and can of course also be used in the securing device according to the invention.

It will also be noted at this point that, as the first and second holders in the context of the present invention, it is also possible to use any desired mechanisms that meet the purpose of the holders, namely that of fixing the prosthesis shaft and the attachment device, respectively.

For example, as regards the prosthesis shaft, a clamping ring that engages around the prosthesis shaft can be provided as holder. The second holder can, for example, be designed in such a way that it achieves fixing by using the securing means, that are present anyway on the attachment device, for the prosthesis and/or the prosthesis adapter.

Particularly advantageously, provision can be made that at least one measuring device is provided for indicating an adjusted relative positioning and/or orientation. Using such measuring devices, it is possible to precisely document which relative position and/or orientation of prosthesis shaft and attachment device was finally chosen in order to create the securing connection. This therefore affords a greatly improved possibility of documentation of the adaptation of the prosthesis parts, which documentation can advantageously be used in further adaptations and/or new prostheses for the patient. In particular, provision can be made that the measuring device is in the form of at least one scale, in particular a scale arranged on a rod guided in a hollow stand and/or on a guide of a clamping ring of a clamping device engaging on a rod and/or at least one scale assigned to an adjustment device for horizontal sliding of the second holder, and/or at least one angle measurer, in particular at least one, in particular two angle measurers arranged on the first holder for the purpose of measuring a pivoting angle about a horizontal axis. It will be noted that many means known in the prior art for relative sliding and/or pivoting already in principle comprise an installed measuring device, which in particular indicates exactly what adjustment has just been made. For example, printed scales, as described, can be provided, or also integrated angle measurers in pivot mechanisms. If a pivoting device is provided as articulated connection, it is particularly advantageously possible to use angle measurers. These can, for example, be provided on the first holder itself. An angle measurer can comprise a plumb-line subject to gravitational force and/or a pointer that can be adjusted to the longitudinal direction of the prosthesis shaft. However, it is also conceivable to replace the plumb-line by a pointer that is to be adjusted manually in the vertical direction. It is then possible to directly read off to what extent the prosthesis shaft is pivoted relative to the vertical.

However, other embodiments are of course also conceivable. In addition to the device, the invention also relates to a method for securing an attachment device for a prosthesis and/or a prosthesis adapter to a prosthesis shaft, for which purpose the device according to the invention can be used particularly advantageously. This method is characterized in that
the attachment device, in a second holder, and the prosthesis shaft, in a first holder, are fixed in a defined relative position and/or orientation to each other,
a final relative position and/or orientation is set by sliding and/or pivoting the holders relative to each other, and
the fixed attachment device and the fixed prosthesis shaft are secured to each other to form a firm connection in the final relative position and/or orientation.

With the method according to the invention, which makes use of a single device to permit suitable adjustment of the relative positioning and/or orientation and to produce the connection, the advantages attainable with the device according to the invent on can be realized. It is stressed here that the observations made concerning the device according to the invention can also be applied analogously to the method according to the invention.

First, both the prosthesis shaft and also the attachment device are clamped in a first holder and a second holder, respectively, of a securing device. Using suitably designed means for relative sliding and/or pivoting of the parts fixed in the holders, the desired relative position and/or orientation can now be adjusted, in particular the desired height and stump setting (abduction and adduction, flexion and extension). When the final relative position and/or orientation that is in particular ideal for the patient is reached, it is then possible, in particular using means likewise provided on the securing device for firm connection of the fixed prosthesis shaft and of the fixed attachment device, to establish a firm connection which secures the attachment device to the prosthesis shaft in the desired final relative position and/or orientation. This therefore permits simple and exact securing, which takes account of the statics of the prosthesis and the individual characteristics of the patient.

In a development of the method, provision can be made that, particularly with the prosthesis shaft fixed in the vertical direction, the first holder, lying above the second holder, is pivoted and/or adjusted in height and/or the second holder is moved in a horizontal plane. The prosthesis shaft can therefore be fixed such that its longitudinal direction corresponds substantially to the vertical and the second holder with the attachment device is positioned underneath. By means of the height adjustment, it is then possible to adapt the height of a subsequent joint, for example a knee joint, and by means of the pivoting, which is advantageously possible about any desired horizontal axis, it is possible to take account of abnormal positioning, for example flexion contractures. Finally, it is also possible to adapt the horizontal relative position of prosthesis shaft and attachment device to the circumstances of the patient, by means of the second holder being movable in a horizontal plane, for example on a base-plate.

Preferably, geometric data describing the final relative position and/or orientation can be measured and/or read off by means of at least one measuring device. Thus, not only is an extremely exact adjustment possible, the latter can also be exactly documented, for example for a subsequent use. Possible examples of such measuring devices are scales that are mounted on mutually slidable parts, for example a scale provided on the base-plate, and/or also angle measurers, as have already been described in respect of the device according to the invention.

A free space lying between the fixed attachment device and the fixed prosthesis shaft can particularly advantageously be filled with a filler, in particular a PU foam, which, in the hardened state, forms at least a part of the connection. As a mold for the filler, it is possible, as has already been described in respect of the device, to use an in particular cylindrical and/or frustoconical, elastic connecting sleeve for sealing the free space and to secure this sleeve in particular to the attachment device, in particular by means of a web corresponding to a groove formed on the attachment device. As is described above, the connecting sleeve can of course also comprise an inlet opening and an air outlet opening, in particular several air outlet openings. However, it is also conceivable, for example, for a hose or the like to be inserted from above, for example between connecting sleeve and prosthesis shaft. Particularly advantageously, the connecting sleeve is turned or rolled over the attachment device and the prosthesis shaft before the sliding and/or pivoting, such that the person carrying out the method is already given a visual clue as to what shape will eventually result. By virtue of the elastic configuration of the connecting sleeve, a change to the relative position and/or orientation is also still possible. As has been described, the connecting sleeve can be cylindrical or shaped as a cone with a slight taper, its lower diameter ideally corresponding to the diameter of the attachment device, which can likewise be round.

The connecting compound used may expediently be a two-component filler, in particular a two-component PU foam, which is mixed in particular in a mixer tube by means of a two-component cartridge and/or in an external container and/or in an injection device. The mixing in the injection device can be achieved, for example, by shaking the latter, although it is also possible for the injection device to comprise a suitable mixer. The two-component filler thus mixed can then be introduced through the inlet opening of the connecting sleeve or in another way, and the air escapes through the air outlet openings or in another way. In principle, however, it is also conceivable to use a one-component filler, for example a one-component PU foam.

After being introduced, the foam hardens inside the free space sealed by the connecting sleeve and thus forms a stable, firm connection, which connects the prosthesis shaft and the attachment device and thus secures the attachment device to the prosthesis shaft.

Provision can particularly advantageously be made for a solvent to be added to the filler in order to improve the adherence of the filler to the attachment device and/or to the prosthesis shaft. The material of the prosthesis shaft and the material of the attachment device are thus lightly etched in the contact area, such that the surface area of adherence is enlarged and, as a result, the adherence is improved.

In an alternative embodiment, provision can be made that those surfaces of the prosthesis shaft and/or of the attachment device that are to be connected to the filler are roughened in order to improve the adherence. In this way too the surface area of adherence is enlarged as a result and the adherence improved, which improves the stability and durability of the arrangement.

If the connecting sleeve is advantageously designed such that there is substantially no adherence or only very slight adherence to the filler, then the connecting sleeve can be removed after the filler has hardened. The connecting sleeve can then be used again in subsequent securing procedures.

Alternatively, however, provision can also be made for the connecting sleeve to remain in place as a protective and/or reinforcing lining and thus form part of the connection. There is then no need to fit another protective lining or to apply a layer, for example by lamination or the like.

In another advantageous embodiment of the method according to the invention, provision can be made that, in order to protect the filler, the latter is provided with a protective lining, in particular a preferably resin-impregnated hose or cone made of a flexible fabric is pulled and/or rolled onto the hardened filler at least, in particular also onto the holder and/or at least part of the prosthesis shaft. Such a protective lining, which, for example, can be fitted after the prosthesis set-up has been checked with the amputee walking and standing, serves in particular to protect the filler, in particular the PU foam, from light, damage and external atmospheric conditions. For this purpose, a resin-impregnated hose or cone made of a flexible fabric is provided in particular. The fabric can be made up of transparent filaments and of components with tensile strength. In particular, the protective lining can be stored in a rolled-up state. The rolled-up protective lining is placed onto the holder from below and unrolled upward, thereby enclosing the hardened filler, but advantageously also the attachment device and at least part of the prosthesis shaft. In this way, the lining also serves to strengthen the connection itself. If the attachment device comprises a groove, which for example has already served to secure the connecting sleeve, provision can be made that the lining is bounded in the groove of the attachment device with an in particular resin-impregnated thread or another thread with tensile strength. As has already been mentioned, this procedure serves not only to protect the filler but also to improve the adherence between prosthesis shaft and attachment device and to increase the strength of the connection.

Provision can also be made that a prosthesis adapter, in particular a modular prosthesis adapter, is mounted on the attachment device. This can be done even before a lining is fitted, in particular after the filler has hardened. For this purpose, the attachment device can comprise, for example, at least one threaded hole, in particular four threaded holes which are used to receive screws, for example. If such threaded holes are continuous, they must of course be closed, for example by a stopper or the like, when the filler is introduced into the free space.

An attachment device provided for use in the device according to the invention or in the method according to the invention comprises attachment means or securing means for attachment of a prosthesis adapter and/or a prosthesis. It advantageously has a substantially round shape and can, on its outer surface, comprise a circumferential groove, which can be used, for example, to secure the connecting sleeve or to subsequently bind a lining.

In order to achieve a better connection by the filler, provision can further be made that, on the side directed toward the prosthesis shaft in the fitted state, the attachment device has at least one upwardly protruding symmetrical and/or asymmetrical web. This enlarges the surface area of adherence. At least one web preferably has an undercut, such that not only is an enlarged surface area of adherence achieved, but also a configuration that advantageously avoids the attachment device simply “dropping out” of the hardened filler.

As has already been mentioned, a surface of the attachment device intended for contact with a filler can be roughened, which means that the surface area of adherence is further enlarged in this way too.

As has also already been mentioned, the attachment means or securing means of the attachment device can comprise at least one threaded hole, in particular four threaded holes. In this way, the prosthesis or the prosthesis adapter can be secured or attached to the attachment device with the aid of screws or the like, for example.

Finally, the invention also relates to a protective lining for a connection, comprising a hardened filler material, of a prosthesis shaft to an attachment device for a prosthesis and/or a prosthesis adapter, which connection is established in particular with the device according to the invention and/or by the method according to the invention. The protective lining according to the invention is characterized in that the in particular hose-shaped or cone-shaped protective lining consisting of an elastic fabric can be pulled, in particular rolled, onto the connection. A protective lining of this kind, which is therefore formed by a fabric hose or fabric cone, can advantageously be made in advance and stored in particular in a rolled-up state. It makes it possible, in a particularly simple way, to protect the filler from external influences, in particular also from light, by means of the protective lining, already present in one piece, being able to be simply pulled, in particular rolled, onto the connection. This is easier to do compared to the known prior art, according to which, for example, a laminate layer is applied or plastic binding is wound round the connection.

Before the protective lining is applied, it is preferably impregnated with resin in order to improve its connecting and protecting properties. Provision can also advantageously be made that it is also dimensioned to cover at least part of the attachment device and/or at least part of the prosthesis shaft. In this way, it not only has the effect of protecting the hardened filler but also strengthens the connection between the prosthesis shaft and the attachment device.

The fabric can preferably be made of transparent filaments in order to provide an improved view.

It will also be noted at this point that the stated attachment device can also be made of a foam, for example.

The securing device according to the invention, in particular the holders, can advantageously be made of metal, although plastic parts are also conceivable.

Further advantages and details of the present invention will become clear from the illustrative embodiments described below and from the drawing, in which:

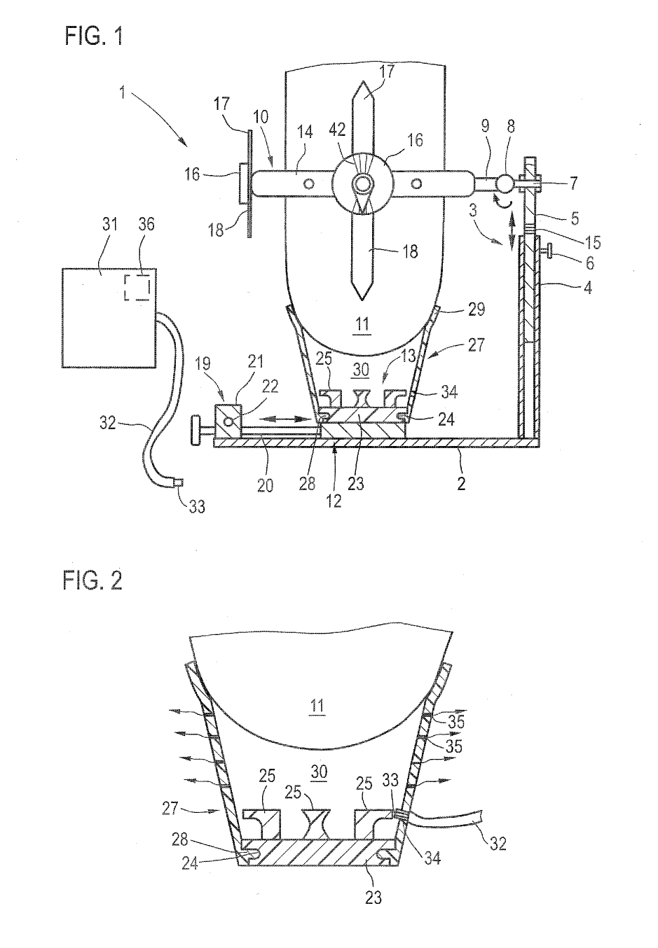

FIG. 1 shows a securing device according to the invention with fixed prosthesis shaft and fixed attachment device,

FIG. 2 shows a diagram detailing the use of a filler,

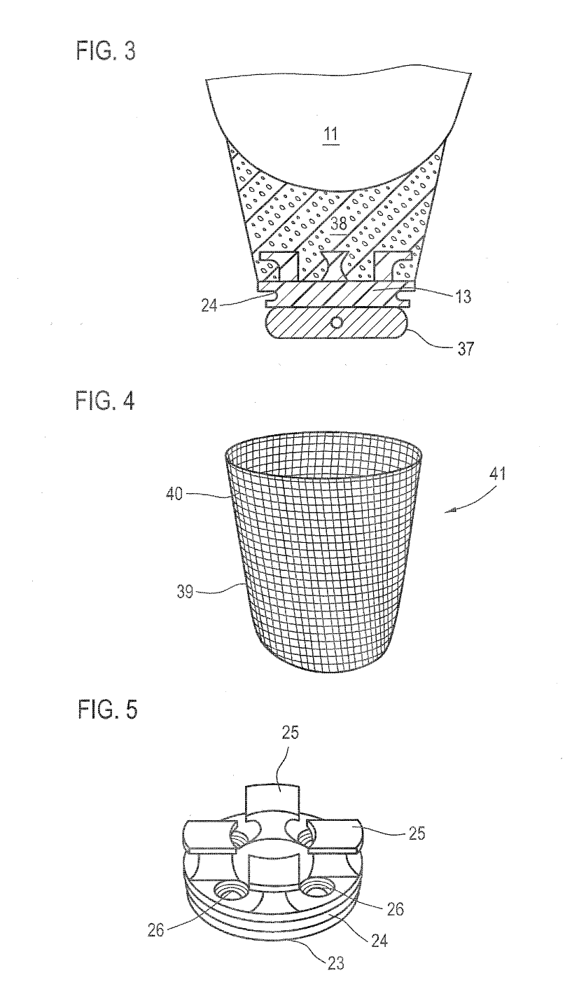

FIG. 3 shows the connection established with hardened filler and with a prosthesis adapter already fitted,

FIG. 4 shows a protective lining according to the invention, and

FIG. 5 shows a perspective view of an attachment device that can be used in the context of the present invention.

An illustrative embodiment of the device according to the invention and an illustrative embodiment of the method according to the invention for securing an attachment device for a prosthesis and/or a prosthesis adapter to a prosthesis shaft are described more or less at the same time hereinbelow in order to illustrate the relationships and the overall procedure.

FIG. 1 shows, partly in cross section, an illustrative embodiment of a securing device 1 according to the invention, in which a prosthesis shaft 11 and an attachment device 13 are already fixed.

The device 1 comprises a metal base-plate 2, on which a stand 3 comprising a hollow tube 4 and a rod 5 guided in the latter are arranged. A locking screw 6, which engages on the rod 5, makes it possible to prevent a movement of the rod 5. The stand 3 thus forms a height-adjusting mechanism. A first support 7 is secured firmly on the rod 5 and is secured, via an articulated connection 8, for example a ball joint, to a second support 9, which is part of a first holder 10 for the prosthesis shaft 11. By means of the height-adjusting mechanism designed as stand 3 and of the articulated connection 8 forming a pivoting device, it is possible to change the height of the first holder 10, and therefore of a prosthesis shaft 11 fixed in the latter, above the base-plate 2 and to pivot the first holder 10 about any desired horizontal axis, such that a prosthesis shaft 11 fixed therein is also tilted. The articulated connection 8 and the stand 3 are therefore part of means for relative sliding and pivoting of the first holder relative to a second holder 12 (discussed in more detail below) guided on the base-plate 2 and provided for the attachment device 13.

The first holder 10 further comprises a ring 14, here a metal ring, which engages with a clamping action around the prosthesis shaft 11.

Moreover, a scale 15 provided on the rod 5 makes it possible to read off the current setting, such that the latter can be documented.

To be able to detect the pivoting via the articulated connection 8, two angle measurers 16, offset by 90°, are mounted on the metal ring 14. These angle measurers 16 each comprise a pointer 17, which is adjusted along the longitudinal axis of the prosthesis shaft, and also a plumb-line 18, which indicates the direction of the gravitational force, i.e. the vertical. The pivoting can be read off on an attached display 42, such that exact documentation is also possible here.

As has already been mentioned, the first holder 10 is arranged above a second holder 12, which is guided on the base-plate 2 and provided for the attachment device 13. On the plate 2, the second holder 12 can be moved in a horizontal plane by an adjustment device 19 of which, for the sake of clarity, only the rod guide is shown here in the direction corresponding to the plane of the drawing. The second holder 12 is consequently arranged on an adjustable rod 20, which can be locked, and which is guided in turn in a slide body 21. The slide body 21 can in turn be guided, via a rod only symbolized here by 22, in the plane perpendicular to the plane of the drawing.

The base-plate 2 is provided with a scale, which allows the instantaneous position of the second holder 12 to be read off and permits documentation.

The first holder 10 and the second holder 12 are therefore coupled to each other via a variety of means for relative sliding and pivoting, namely the adjustment mechanism 19, the stand 3 and the articulated connection 8. The current setting can be established or read off for the purpose of documentation via measuring devices, namely the scale 15, the angle measurers 16 and the scale (not shown here) on the base-plate 2, for the purpose of documentation.

A description of the used attachment device 13 is included at this point (cf. also FIG. 5). This attachment device 13 is basically formed by a round plastic plate 23 which, on its outer surface, comprises a circumferential groove 24. Moreover, several symmetrical webs 25, which are undercut, are integrally formed on the side directed toward the prosthesis shaft 11. These webs 25 serve for a better, form-fit connection to a filler, which will be discussed in more detail below. Finally, the attachment device 13 has threaded holes 26 (only visible in FIG. 5), which later serve for securing a prosthesis adapter and/or a prosthesis. In the present case, some of the threaded holes 26 are closed by stoppers and some serve for fixing the attachment device 13 to the second holder 12, for which purpose corresponding screw holders or clamp holders can be provided on the upper face of the holder 12. It will be noted at this point that the attachment device 13 can also be secured to the second holder 12 in another way, for example by means of a releasable adhesive or another clamping device, and it is in particular also conceivable to provide securing only through static friction.

The securing device 1 further comprises a connecting sleeve 27 made of a flexible material and having a conically tapering shape and a web 28 provided at its lower end, which web 28 corresponds to the groove 24 and serves to temporarily secure the connecting sleeve 27 to the attachment device 13.

Once secured, the connecting sleeve 27, bearing with its upper ends 29 on the prosthesis shaft 11, consequently seals off a free space 30 between the prosthesis shaft 11 and the attachment device 13, such that this free space can be filled with a filler, which then hardens. Therefore, the connecting sleeve 27 also has the shape that the filler, forming part of the resulting firm connection, is later intended to adopt.

The connecting sleeve 27 can, for example, be folded open on one side such that it can be suitably pushed over, in which case closure means can be provided in order also to seal it there. The flexibility of the connecting sleeve 27 makes it possible, even when the connecting sleeve 27 is already fitted, to still permit a relative sliding and/or pivoting of the parts fixed in the holders, that is to say of the prosthesis shaft 11 and the attachment device 13.

The height of the connecting sleeve 27 is to be chosen such that it is suitable for different heights set via the stand 3.

To be able to introduce the filler into the free space 30, the device 1 further comprises an injection device 31 with a hose 32 attached thereto, which hose 32 ends in a nozzle 33. The nozzle 33 can be pushed into an inlet opening 34 of the connecting sleeve 27, in which respect reference is made specifically to FIG. 2.

It will be seen there that the free space 30 is delimited at the top by the prosthesis shaft 11, at the bottom by the attachment device 13, and at the sides by the connecting sleeve 27. The nozzle 33 of the hose 32 is inserted through the inlet opening 34, and, in the present case, the inlet opening 34 is provided with a closure device (not shown), which is opened when the nozzle 33 is inserted, and which again closes the inlet opening 34 when the nozzle 33 is withdrawn.

To let the air out during the injection of the filler, the connecting sleeve 27 also has air outlet openings 35, of which the size has been chosen such that air can escape from the free space 30 while the latter is being filled, but such that they do not allow the filler to pass through them, and instead the filler simply closes the air outlet openings 35 when the appropriate filling level is reached. The air outlet openings 35 are provided in circles at different heights and are therefore distributed substantially uniformly.

It will be noted at this point that other solutions can of course also be provided for injecting the filler and for letting the air out. For example, the outlet openings 35 can first open into a channel which extends inside the wall of the connecting sleeve 27 and which, at another location, has an opening to the environment. It is also conceivable, both for the injection and also for the removal of the escaping air, to insert hoses from above in between the prosthesis shaft 11 and the connecting sleeve 27.

The method according to the invention can now advantageously be carried out with the device 1 described. For this purpose, as is also already shown in the figures, the prosthesis shaft 11 is first fixed in the first holder 10 and the attachment device 13 in the second holder 12. The connecting sleeve 27 is then secured to the attachment device 13, in which respect it will be noted that it is also possible, particularly in the case of a connecting sleeve 27 that is closed all the way round, for the connecting sleeve 27 to be pushed onto the attachment device 13 beforehand, that is to say before the prosthesis shaft 11 is inserted into it from above and fixed. This fixing can, for example, take place in a starting position of the holders such that the longitudinal direction of the prosthesis shaft 11 corresponds exactly to the vertical, and the mid-line of the prosthesis shaft 11 intersects the mid-point of the round attachment device 13.

Thereafter, with the aid of the means for relative sliding and pivoting, i.e. by means of the stand 3, the articulated connection 8 and the adjustment mechanism 19, it is possible to change the relative position and orientation of the prosthesis shaft 11 and of the attachment device 13 until it is ideal for the patient and a final position and orientation has been chosen. The measuring devices, that is to say the scale 15, the angle measurers 16, and the scale provided on the base-plate 2, are used to measure and read off geometric data describing the final position and orientation, such that documentation can take place.

While the prosthesis shaft 11 in the first holder 10 and the attachment device 13 in the second holder 12 are held in the final relative position and orientation, the firm connection is now made ready. In the present example, the filler used is a two-component PU foam, which is initially mixed in the mixer tube by means of a two-component cartridge and which, by means of the injection device 31 and the hose 32, with its nozzle 33 inserted into the inlet opening 34, is injected into the free space 30 delimited all round by the prosthesis shaft 11, the attachment device 13 and the connecting sleeve 27. Provision can also be made that the PU foam is mixed in a separate container and then introduced into the injection device 31 or is mixed by vibration of the injection device 31. In addition, the injection device 31 can also contain a mixer mechanism, indicated as an option at 36.

To improve the adherence of the filler, that is to say of the two-component PU foam, to the material of the prosthesis shaft 11 and to the material of the attachment device 13, a solvent is admixed to the PU foam and lightly etches the materials of the prosthesis shaft 11 and of the attachment device 13 and thus increases the surface area of adherence. The connecting sleeve 27, however, remains unaffected. It will be noted that, as an alternative to the addition of the solvent, the corresponding surface of the prosthesis shaft 11 and of the attachment device 13 can also be slightly roughened.

During injection, the filler displaces the air in the free space 30, and this air escapes through the small air outlet openings 35 of the connecting sleeve 27. When the filler has reached substantially the upper edge of the free space 30, the injection device 31 can be detached from the connecting sleeve 27. The inlet opening closes automatically. If a functionality of this kind is not provided, the hose has to be squeezed shut.

After the filler has hardened, the connecting sleeve 27 can be detached, since it does not bind to the polyurethane foam. The connecting sleeve 27 can then be used again in a further procedure, although it is also possible to provide one connecting sleeve 27 for each procedure.

Thereafter, the prosthesis adapter 37 can be mounted on the attachment device 13, as is shown in FIG. 3. The threaded holes 26 are used to secure the prosthesis adapter 37. The hardened filler 38 still lies open at this point.

By virtue of the modular prosthesis adapter 37, the other prosthesis components can now also be attached, and the prosthesis setting can be checked while the amputee is walking and standing.

Finally, it is also possible to protect the hardened filler 38, i.e. the PU foam, from light, damage and external atmospheric conditions. For this purpose, a lining is used, as is shown in the schematic diagram in FIG. 4.

The protective lining 41 is designed as a conically tapering hose or as a cone 39 made of a flexible fabric 40. The threads of the fabric 40 can be transparent. The resin-impregnated protective lining 41 is placed in a rolled-up state under the attachment device 13 and unrolled upward thereon. In so doing, it encloses the attachment device 13, the hardened filler 38 and part of the prosthesis shaft 11. The groove 24 of the attachment device 13 is now used to bind the lining 41 therein with a resin-impregnated thread or another thread with tensile strength. This serves not only to protect the PU foam but also to improve the adherence between prosthesis shaft 11 and attachment device 13 and also to increase the strength of the resulting connection, which in the present example is then formed by the hardened filler 38 and the lining 41.

1. Device for securing an attachment device for a prosthesis and/or a prosthesis adapter to a prosthesis shaft, comprising:
a first holder for the prosthesis shaft,
a second holder for the attachment device, said second holder being arranged in particular below the first holder,
means for relative sliding and/or pivoting of the prosthesis shaft fixed in the first holder and of the attachment device fixed in the second holder, and
means for firmly connecting the fixed prosthesis shaft and the fixed attachment device in a desired relative position and/or orientation.

2. Device according to claim 1, wherein the means for the firm connection comprise a flexible connecting sleeve that can be secured in particular on the attachment device, in particular by means of a web corresponding to a groove of the attachment device, and that seals off the free space between the prosthesis shaft fixed in the first holder and the attachment device fixed in the second holder.

3. Device according to claim 2, wherein the connecting sleeve has a configuration corresponding to the desired final shape of the connection, in particular a frustoconical shape and/or a cylindrical shape, and/or is made of a material that does not undergo any appreciable adhesive bonding to a filler.

4. Device according to claim 1, wherein the means for the relative pivoting and/or sliding comprise a height-adjusting mechanism for the first holder and/or at least one pivoting device for the first holder.

5. Device according to claim 1, wherein at least one measuring device is provided for indicating an adjusted relative positioning and/or orientation.

6. Method for securing an attachment device for a prosthesis and/or a prosthesis adapter to a prosthesis shaft, in particular using a device according to claim 1, wherein
the attachment device, in a second holder, and the prosthesis shaft, in a first holder, are fixed in a defined relative position and/or orientation to each other,
a final relative position and/or orientation is set by sliding and/or pivoting the holders relative to each other, and
the fixed attachment device and the fixed prosthesis shaft are secured to each other to form a firm connection in the final relative position and/or orientation.

7. Method according to claim 6, wherein the first holder, lying above the second holder, is pivoted and/or adjusted in height and/or the second holder is moved in a horizontal plane.

8. Method according to claim 6, wherein geometric data describing the final relative position and/or orientation are measured and/or read off by means of at least one measuring device.

9. Method according to claim 6, wherein a free space lying between the fixed attachment device and the fixed prosthesis shaft is filled with a filler, in particular a PU foam, which, in the hardened state, forms at least a part of the connection.

10. Method according to claim 9, wherein, as a mold for the filler, an in particular cylindrical and/or frustoconical, elastic connecting sleeve is used for sealing the free space and is secured in particular to the attachment device, in particular by means of a web corresponding to a groove formed on the attachment device.

11. Method according to claim 9, wherein, in order to protect the filler, the latter is provided with a protective lining, in particular a preferably resin-impregnated hose or cone made of a flexible fabric is rolled onto the hardened filler at least, in particular also onto the holder and/or at least part of the prosthesis shaft.

12. Method according to one of claim 6, wherein a prosthesis adapter, in particular a modular prosthesis adapter, is mounted on the attachment device.

13. Protective lining for a connection, comprising a hardened filler material, of a prosthesis shaft to an attachment device for a prosthesis and/or a prosthesis adapter, which connection has been established in particular with the device according to claim 1, wherein the in particular hose-shaped or cone-shaped protective lining consisting of an elastic fabric can be pulled, in particular rolled, onto the connection.

14. Protective lining according to claim 13, wherein it is also dimensioned to cover at least part of the attachment device and/or at least part of the prosthesis shaft.
